(19) United States
(12) Patent Application Publication
(10) Pub. No.: US 2024/0285195 A1
(43) Pub. Date: Aug. 29, 2024

(54) NON-INVASIVE ANALYTE SENSOR WITH MOTION AND/OR TEMPERATURE SENSOR

(71) Applicant: Know Labs, Inc., Seattle, WA (US)

(72) Inventors: Steve LOWE, Seattle, WA (US); Steven KENT, Seattle, WA (US)

(21) Appl. No.: 18/585,483

(22) Filed: Feb. 23, 2024

Related U.S. Application Data
(60) Provisional application No. 63/486,780, filed on Feb. 24, 2023.

(51) Int. Cl. A61B 5/145; A61B 5/00; A61B 5/01; A61B 5/0507; A61B 5/11

(52) U.S. Cl. CPC A61B 5/14546; A61B 5/01; A61B 5/0507; A61B 5/11; A61B 5/1451; A61B 5/7221; A61B 2560/0252; A61B 2562/0219; A61B 2562/0271

(57) ABSTRACT

A non-invasive analyte sensor includes one or more motion sensors and/or one or more temperature sensors. Data from the one or more motion sensors and/or one or more temperature sensors can be used to post-process the data obtained by the non-invasive analyte sensor.

### FIELD

This disclosure relates generally to apparatus, systems and methods of detecting an analyte via spectroscopic techniques using an analyte sensor that includes a detector array (also referred to as an antenna array), wherein the detector array operates in the radio or microwave frequency range of the electromagnetic spectrum.

### BACKGROUND

There is interest in being able to detect and/or measure an analyte within a target. One example is measuring glucose in biological material. In the example of measuring glucose in a patient, current analyte measurement methods are invasive in that they perform the measurement on a bodily fluid such as blood for fingerstick or laboratory-based tests, or on fluid that is drawn from the patient often using an invasive transcutaneous device. There are non-invasive methods that claim to be able to perform glucose measurements in biological material. However, many of the non-invasive methods generally suffer from: lack of specificity to the analyte of interest, such as glucose; interference from temperature fluctuations; interference from skin compounds (i.e. sweat) and pigments; and complexity of placement, i.e. the sensing device resides on multiple locations on the patient's body.

### SUMMARY

This disclosure relates generally to apparatus, systems and methods of detecting an analyte via spectroscopic techniques using non-optical frequencies such as in the radio or microwave frequency range of the electromagnetic spectrum. An analyte sensor described herein includes a detector array having a plurality of detector elements (also referred to as antenna elements or antennas) at least one of which can transmit an electromagnetic signal in the radio or microwave frequency range and at least one of which can receive an electromagnetic signal in the radio or microwave frequency range resulting from transmission of the electromagnetic signal.

In the non-invasive analyte sensor described herein, the sensor receives data from at least one motion sensor and/or temperature sensor. The motion sensor and/or temperature sensor may be integrated with the analyte sensor, or separate from but connected to the analyte sensor.

In embodiments, a non-invasive analyte sensor system can include an antenna that is configured to detect an electromagnetic wave in a radio or microwave frequency range that results from transmission of a transmit signal in the radio or microwave frequency range into a target containing at least one analyte, a receive circuit that is electrically connectable to the antenna to convert the electromagnetic wave into one or more signals representing the at least one analyte and a motion sensor.

In some embodiments, the motion sensor comprises an accelerometer, a gyroscope, or an inertial measurement unit.

In some embodiments, the at least one analyte comprises glucose, alcohol, white blood cells, oxygen, or luteinizing hormone.

In some embodiments, the system further includes a housing configured to contain the motion sensor.

In some embodiments, the motion sensor is configured to sense motion of the housing relative to the target.

In some embodiments, the motion sensor is configured to sense motion of the housing together with the target.

In some embodiments, the system further includes a controller configured to process analyte data contained in the one or more signals representing the at least one analyte in view of motion data obtained by the motion sensor.

In some embodiments, the controller is configured to process the analyte data to filter analyte data obtained at a time of movement based on the motion data at the time of movement.

In some embodiments, the controller is configured to process the analyte data to determine that an analyte measurement should not be utilized based on the motion data.

In some embodiments, the controller is configured to determine that an analyte measurement should not be utilized if it is determined that the analyte measurement is too inaccurate based on the motion data.

In some embodiments, the system further includes a controller configured to utilize motion data obtained by the motion sensor to trigger an analyte measurement using the antenna.

In some embodiments, the system further includes a controller configured to utilize motion data obtained by the motion sensor to prevent an analyte measurement using the antenna.

In embodiments, a non-invasive analyte sensor system can include an antenna that is configured to detect an electromagnetic wave in a radio or microwave frequency range that results from transmission of a transmit signal in the radio or microwave frequency range into a target containing at least one analyte, a receive circuit that is electrically connectable to the antenna to convert the electromagnetic wave into one or more signals representing the at least one analyte and a temperature sensor.

In some embodiments, the temperature sensor is configured to sense a temperature of one or more of the antenna and the receive circuit.

In some embodiments, the temperature sensor is configured to sense a temperature of the target.

In some embodiments, the temperature sensor is configured to sense a temperature of the at least one analyte.

In some embodiments, the temperature sensor is configured to sense an ambient temperature.

In some embodiments, the system further includes a controller configured to adjust operation of the antenna based on temperature data obtained by the temperature sensor.

In some embodiments, the system further includes a controller configured to process the one or more signals representing the at least one analyte based on temperature data obtained by the temperature sensor.

In some embodiments the controller is configured to process the one or more signals representing the at least one analyte based on the temperature data obtained by the temperature sensor to account for temperature-related signal drift.

### BRIEF DESCRIPTION OF DRAWINGS

FIG. 1 is a schematic depiction of an analyte sensor system with an analyte sensor relative to a target according to an embodiment.

FIG. 2 is a schematic depiction of an embodiment of a non-invasive analyte sensor system with an antenna array having three antennas.

FIG. 3 is a schematic depiction of another embodiment of a non-invasive analyte sensor system with an antenna array having six antennas.

FIG. 4 is a schematic depiction of an embodiment of a circuit that can be used in the analyte sensor.

Like reference numbers represent like parts throughout.

### DETAILED DESCRIPTION

The following is a detailed description of apparatus, systems and methods of detecting an analyte via spectroscopic techniques using non-optical frequencies such as in the radio or microwave frequency bands of the electromagnetic spectrum. An analyte sensor described herein includes a detector array having a plurality of detector elements (also referred to as antenna elements or antennas) at least one of which can transmit an electromagnetic signal in the radio or microwave frequency range and at least one of which can receive an electromagnetic signal in the radio or microwave frequency range resulting from transmission of the electromagnetic signal. For sake of convenience, the detector array will hereinafter be referred to as an antenna array and the detector elements will hereinafter be referred to as antennas. In an embodiment, the analyte sensor can include a single antenna that functions to both transmit signals and receive returning signals that result from the transmitted signals.

In one embodiment, the sensor systems described herein can be used to detect the presence of at least one analyte in a target. In another embodiment, the sensor systems described herein can detect an amount or a concentration of the at least one analyte in the target. The target can be any target containing at least one analyte of interest that one may wish to detect. The target can be human or non-human, animal or non-animal, biological or non-biological. For example, the target can include, but is not limited to, human tissue, animal tissue, plant tissue, an inanimate object, soil, a fluid, genetic material, or a microbe. Non-limiting examples of targets include, but are not limited to, a fluid, for example blood, interstitial fluid, cerebral spinal fluid, lymph fluid or urine, human tissue, animal tissue, plant tissue, an inanimate object, soil, genetic material, or a microbe. In one embodiment, the non-invasive analyte sensor described herein can simultaneously detect an analyte in both blood and in interstitial fluid. In another embodiment, the non-invasive analyte sensor described herein can simultaneously detect an analyte in blood, in interstitial fluid, and cellular material. In addition, the non-invasive analyte sensor described herein can be used without requiring any specific alignment on the human body. For example, the non-invasive analyte sensor described herein does not need to be aligned with a vein or an artery during sensing of an analyte.

The detection by the sensors described herein can be non-invasive meaning that the sensor remains outside the target, such as the human body, and the detection of the analyte occurs without requiring removal of fluid or other removal from the target, such as the human body. In the case of sensing in the human body, this non-invasive sensing may also be referred to as in vivo sensing. In other embodiments, the sensors described herein may be an in vitro sensor where the material containing the analyte has been removed, for example from a human body.

The transmit antenna and the receive antenna can be located near the target and operated as further described herein to assist in detecting at least one analyte in the target. The transmit antenna transmits a signal that is in the radio or microwave frequency range, toward and into the target. The receive antenna detects a response resulting from transmission of the signal by the transmit antenna into the target containing the at least one analyte of interest.

The transmit antenna and the receive antenna may be decoupled (which may also be referred to as detuned or the like) from one another. Decoupling refers to intentionally fabricating the configuration and/or arrangement of the transmit antenna and the receive antenna to minimize direct communication between the transmit antenna and the receive antenna, preferably absent shielding. Shielding between the transmit antenna and the receive antenna can be utilized. However, the transmit antenna and the receive antenna are decoupled even without the presence of shielding.

The signal(s) detected by the receive antenna can be analyzed to detect the analyte based on the intensity of the received signal(s) and reductions in intensity at one or more frequencies where the analyte absorbs the transmitted signal. Examples of detecting an analyte using a non-invasive spectroscopy sensor operating in the radio or microwave frequency range of the electromagnetic spectrum are described in U.S. Pat. Nos. 10,548,503, 11,529,077, 11,063,373, 11,058,331, 11,033,208, 11,284,819, 11,284,820, 10,548,503, 11,234,619, 11,031,970, 11,223,383, 11,058,317, 11,193,923, 11,234,618, 11,389,091, U.S. 2021/0259571, U.S. 2022/0077918, U.S. 2022/0071527, U.S. 2022/0074870, and U.S. 2022/0151553 which are incorporated herein by reference in their entirety.

The analyte(s) can be any analyte that one may wish to detect. The analyte can be human or non-human, animal or non-animal, biological or non-biological. For example, the analyte(s) can include, but is not limited to, one or more of glucose, alcohol, white blood cells, luteinizing hormone, one or more analytes indicative of an oxygen level of a subject, such as, for example, elemental oxygen, oxyhemoglobin, deoxyhemoglobin, and/or any other suitable indicator or proxy for an oxygen level in the subject. The analyte(s) can include, but is not limited to, a chemical, a combination of chemicals, a virus, a bacteria, or the like. The analyte can be a chemical included in another medium, with non-limiting examples of such media including a fluid containing the at least one analyte, for example blood, interstitial fluid, cerebral spinal fluid, lymph fluid or urine, human tissue, animal tissue, plant tissue, an inanimate object, soil, genetic material, or a microbe. The analyte(s) may also be a non-human, non-biological particle such as a mineral or a contaminant.

The analyte(s) can include, for example, naturally occurring substances, artificial substances, metabolites, and/or reaction products. As non-limiting examples, the at least one analyte can include, but is not limited to, insulin, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; pro-BNP; BNP; troponin; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, analyte-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; analyte-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, polio virus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urca; uroporphyrinogen I synthase; vitamin A; white blood cells; and zinc protoporphyrin.

The analyte(s) can also include one or more chemicals introduced into the target. The analyte(s) can include a marker such as a contrast agent, a radioisotope, or other chemical agent. The analyte(s) can include a fluorocarbon-based synthetic blood. The analyte(s) can include a drug or pharmaceutical composition, with non-limiting examples including ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The analyte(s) can include other drugs or pharmaceutical compositions. The analyte(s) can include neurochemicals or other chemicals generated within the body, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA).

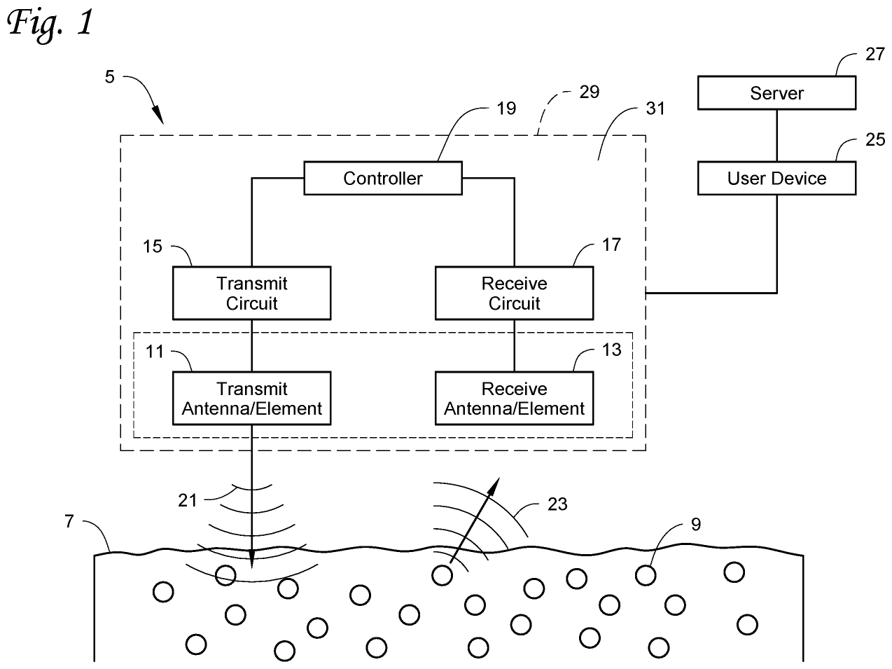

Referring now to FIG. 1, an embodiment of a non-invasive analyte sensor system with a non-invasive analyte sensor 5 is illustrated. The sensor 5 is depicted relative to a target 7 that contains an analyte of interest 9, for example an analyte in interstitial fluid in a human body. In this example, the sensor 5 is depicted as including an antenna array that includes a transmit antenna/element 11 (hereinafter “transmit antenna 11”) and a receive antenna/element 13 (hereinafter “receive antenna 13”). The sensor 5 further includes a transmit circuit 15, a receive circuit 17, and a controller 19. As discussed further below, the sensor 5 can also include a power supply, such as a battery (not shown in FIG. 1). In some embodiments, power can be provided from mains power, for example by plugging the sensor 5 into a wall socket via a cord connected to the sensor 5.

The transmit antenna 11 is positioned, arranged and configured to transmit a signal 21 that is in the radio frequency (RF) or microwave range of the electromagnetic spectrum into the target 7. In an embodiment, the transmitted signals can have a frequency in the range from about 400 MHz to about 4400 MHz. In another embodiment, the transmitted signals can have a frequency in the range from about 500 MHz to about 3000 MHz. The transmit antenna 11 can be an electrode or any other suitable transmitter of electromagnetic signals in the radio frequency (RF) or microwave range. The transmit antenna 11 can have any arrangement and orientation relative to the target 7 that is sufficient to allow the analyte sensing to take place. In one non-limiting embodiment, the transmit antenna 11 can be arranged to face in a direction that is substantially toward the target 7.

The signal 21 transmitted by the transmit antenna 11 is generated by the transmit circuit 15 which is electrically connectable to the transmit antenna 11. The transmit circuit 15 can have any configuration that is suitable to generate a transmit signal to be transmitted by the transmit antenna 11. Transmit circuits for generating transmit signals in the RF or microwave frequency range are well known in the art. In one embodiment, the transmit circuit 15 can include, for example, a connection to a power source, a frequency generator, and optionally filters, amplifiers or any other suitable clements for a circuit generating an RF or microwave frequency electromagnetic signal.

The receive antenna 13 is positioned, arranged, and configured to detect one or more electromagnetic response signals 23 that result from the transmission of the transmit signal 21 by the transmit antenna 11 into the target 7 and impinging on the analyte 9. The receive antenna 13 can be an electrode or any other suitable receiver of electromagnetic signals in the radio frequency (RF) or microwave range. The receive antenna 13 can have any arrangement and orientation relative to the target 7 that is sufficient to allow detection of the response signal(s) 23 to allow the analyte sensing to take place. In one non-limiting embodiment, the receive antenna 13 can be arranged to face in a direction that is substantially toward the target 7.

The receive circuit 17 is electrically connectable to the receive antenna 13 and conveys the received response from the receive antenna 13 to the controller 19. The receive circuit 17 can have any configuration that is suitable for interfacing with the receive antenna 13 to convert the electromagnetic energy detected by the receive antenna 13 into one or more signals reflective of the response signal(s) 23. The construction of receive circuits are well known in the art. The receive circuit 17 can be configured to condition the signal(s) prior to providing the signal(s) to the controller 19, for example through amplifying the signal(s), filtering the signal(s), or the like. Accordingly, the receive circuit 17 may include filters, amplifiers, or any other suitable components for conditioning the signal(s) provided to the controller 19.

The controller 19 controls the operation of the sensor 5. The controller 19, for example, can direct the transmit circuit 15 to generate a transmit signal to be transmitted by the transmit antenna 11. The controller 19 further receives signals from the receive circuit 17. The controller 19 can optionally process the signals from the receive circuit 17 to detect the analyte(s) 9 in the target 7. In one embodiment, the controller 19 may optionally be in communication with at least one external device 25 such as a user device and/or a remote server 27, for example through one or more wireless connections such as Bluetooth, wireless data connections such a 4G, 5G, LTE or the like, or Wi-Fi. If provided, the external device 25 and/or remote server 27 may process (or further process) the signals that the controller 19 receives from the receive circuit 17, for example to detect the analyte(s) 9. If provided, the external device 25 may be used to provide communication between the sensor 5 and the remote server 27, for example using a wired data connection or via a wireless data connection or Wi-Fi of the external device 25 to provide the connection to the remote server 27.

With continued reference to FIG. 1, the sensor 5 may include a sensor housing 29 (shown in dashed lines) that defines an interior space 31. Components of the sensor 5 may be attached to and/or disposed within the housing 29. For example, the transmit antenna 11 and the receive antenna 13 are attached to the housing 29. In some embodiments, the antennas 11, 13 may be entirely or partially within the interior space 31 of the housing 29. In some embodiments, the antennas 11, 13 may be attached to the housing 29 but at least partially or fully located outside the interior space 31. In some embodiments, the transmit circuit 15, the receive circuit 17 and the controller 19 are attached to the housing 29 and disposed entirely within the sensor housing 29.

The receive antenna 13 may be decoupled or detuned with respect to the transmit antenna 11 such that electromagnetic coupling between the transmit antenna 11 and the receive antenna 13 is reduced. The decoupling of the transmit antenna 11 and the receive antenna 13 increases the portion of the signal(s) detected by the receive antenna 13 that is the response signal(s) 23 from the target 7, and minimizes direct receipt of the transmitted signal 21 by the receive antenna 13.

In an embodiment, coupling between the transmit antenna 11 and the receive antenna 13 is 95% or less. In another embodiment, coupling between the transmit antenna 11 and the receive antenna 13 is 90% or less. In another embodiment, coupling between the transmit antenna 11 and the receive antenna 13 is 85% or less. In another embodiment, coupling between the transmit antenna 11 and the receive antenna 13 is 75% or less.

Any technique for reducing coupling between the transmit antenna 11 and the receive antenna 13 can be used. For example, the decoupling between the transmit antenna 11 and the receive antenna 13 can be achieved by one or more intentionally fabricated configurations and/or arrangements between the transmit antenna 11 and the receive antenna 13 that is sufficient to decouple the transmit antenna 11 and the receive antenna 13 from one another.

For example, in one embodiment described further below, the decoupling of the transmit antenna 11 and the receive antenna 13 can be achieved by intentionally configuring the transmit antenna 11 and the receive antenna 13 to have different geometries from one another. Intentionally different geometries refers to different geometric configurations of the transmit and receive antennas 11, 13 that are intentional. Intentional differences in geometry are distinct from differences in geometry of transmit and receive antennas that may occur by accident or unintentionally, for example due to manufacturing errors or tolerances.

Another technique to achieve decoupling of the transmit antenna 11 and the receive antenna 13 is to provide appropriate spacing between each antenna 11, 13 that is sufficient to decouple the antennas 11, 13 and force a proportion of the electromagnetic lines of force of the transmitted signal 21 into the target 7 thereby minimizing or eliminating as much as possible direct receipt of electromagnetic energy by the receive antenna 13 directly from the transmit antenna 11 without traveling into the target 7. The appropriate spacing between each antenna 11, 13 can be determined based upon factors that include, but are not limited to, the output power of the signal from the transmit antenna 11, the size of the antennas 11, 13, the frequency or frequencies of the transmitted signal, and the presence of any shielding between the antennas. This technique helps to ensure that the response detected by the receive antenna 13 is measuring the analyte 9 and is not just the transmitted signal 21 flowing directly from the transmit antenna 11 to the receive antenna 13. In some embodiments, the appropriate spacing between the antennas 11, 13 can be used together with the intentional difference in geometries of the antennas 11, 13 to achieve decoupling.

In one embodiment, the transmit signal (or each of the transmit signals) can be transmitted over a transmit time that is less than, equal to, or greater than about 300 ms. In another embodiment, the transmit time can be than, equal to, or greater than about 200 ms. In still another embodiment, the transmit time can be less than, equal to, or greater than about 30 ms. The transmit time could also have a magnitude that is measured in seconds, for example 1 second, 5 seconds, 10 seconds, or more. In an embodiment, the same transmit signal can be transmitted multiple times, and then the transmit time can be averaged. In another embodiment, the transmit signal (or cach of the transmit signals) can be transmitted with a duty cycle that is less than or equal to about 50%.

The interaction between the transmitted signal and the analyte may, in some cases, increase the intensity of the signal(s) that is detected by the receive antenna, and may, in other cases, decrease the intensity of the signal(s) that is detected by the receive antenna. For example, in one non-limiting embodiment, when analyzing the detected response, compounds in the target, including the analyte of interest that is being detected, can absorb some of the transmit signal, with the absorption varying based on the frequency of the transmit signal. The response signal detected by the receive antenna may include drops in intensity at frequencies where compounds in the target, such as the analyte, absorb the transmit signal. The frequencies of absorption are particular to different analytes. The response signal(s) detected by the receive antenna can be analyzed at frequencies that are associated with the analyte of interest to detect the analyte based on drops in the signal intensity corresponding to absorption by the analyte based on whether such drops in signal intensity are observed at frequencies that correspond to the absorption by the analyte of interest. A similar technique can be employed with respect to increases in the intensity of the signal(s) caused by the analyte.

Detection of the presence of the analyte can be achieved, for example, by identifying a change in the signal intensity detected by the receive antenna at a known frequency associated with the analyte. The change may be a decrease in the signal intensity or an increase in the signal intensity depending upon how the transmit signal interacts with the analyte. The known frequency associated with the analyte can be established, for example, through testing of solutions known to contain the analyte. Determination of the amount of the analyte can be achieved, for example, by identifying a magnitude of the change in the signal at the known frequency, for example using a function where the input variable is the magnitude of the change in signal and the output variable is an amount of the analyte. The determination of the amount of the analyte can further be used to determine a concentration, for example based on a known mass or volume of the target. In an embodiment, presence of the analyte and determination of the amount of analyte may both be determined, for example by first identifying the change in the detected signal to detect the presence of the analyte, and then processing the detected signal(s) to identify the magnitude of the change to determine the amount.

In another embodiment, detection of the presence of the analyte can be achieved by comparing the return signals from the receive antenna with ground truth data. For example, in the case of the analyte being glucose, the ground truth data can be reference glucose readings obtained by one or more reference glucose sensors, such as a minimally invasive glucose sensor such as a Dexcom G6 continuous glucose monitor.

Further information on the sensor 5 and its components and variations thereof can be found in U.S. Pat. Nos. 11,063,373, 11,031,970, 11,058,317, 11,058,331 and 11,033,208 the entire contents of which are incorporated herein by reference in their entirety.

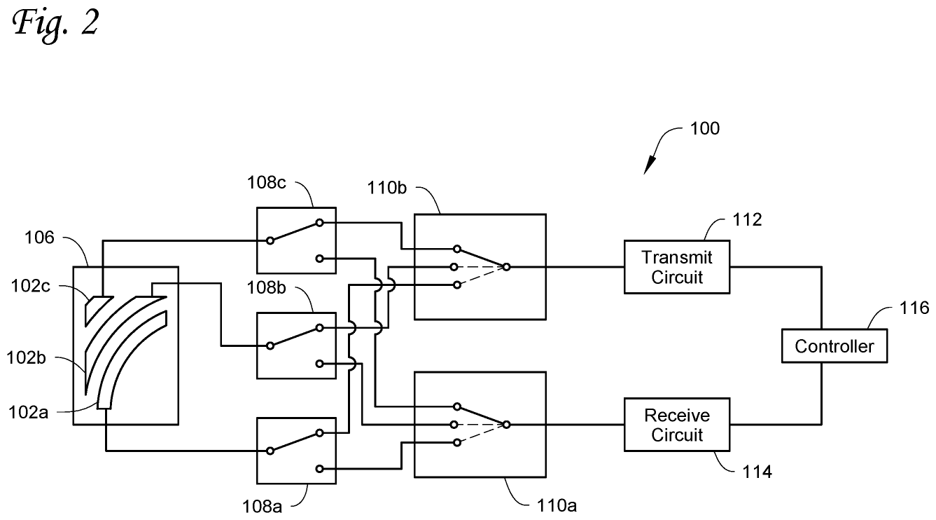

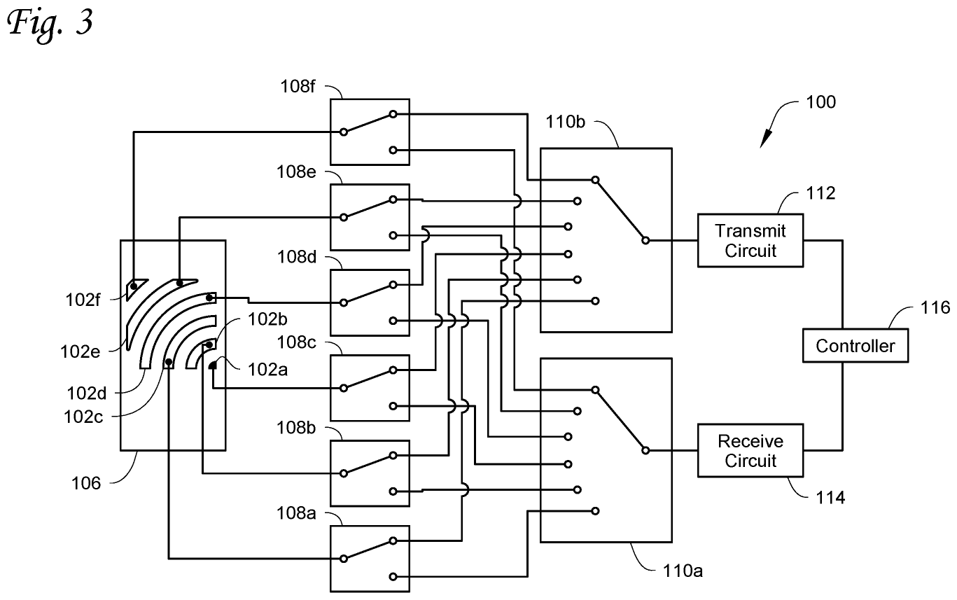

FIGS. 2-3 are schematic depictions of additional embodiments of a non-invasive analyte sensor system 100. The systems 100 depicted in FIGS. 2-3 includes at least three or more antennas (FIG. 2) or at least six or more antennas (FIG. 3). However, a different number of antennas can be used. In each of the embodiments, the system 100 is configured so that one or more of the antennas of the antenna array can be used as either a transmit antenna or as a receive antenna. In FIGS. 2-3, like elements are referenced using the same reference numerals. As with the previously described embodiment in FIG. 1, the antenna arrays in FIGS. 2-3 can be a decoupled antenna array and the antennas of the antenna array can be decoupled from one another. However, in some embodiments, the antennas of the system 100 may not be decoupled from one another. In one embodiment, the antennas used in the arrays in FIGS. 2-3 can have different geometries from each other.

In the embodiment in FIG. 2, the antenna array of the system 100 has three antennas 102a, 102b, 102c each of which is disposed on a substrate 106. The system further includes three switches 108a, 108b, 108c, a receive switch controller 110a, a transmit switch controller 110b separate from the receive switch controller 110a, a transmit circuit 112, a receive circuit 114, and a controller 116. In the embodiment in FIG. 3, the antenna array of the system 100 has six antennas 102a-f each of which is disposed on the substrate 106, and six of the switches 108a-f. Further information on the system 100 in FIGS. 2 and 3 can be found in U.S. Pat. No. 11,058,321, the entire contents of which are incorporated herein by reference.

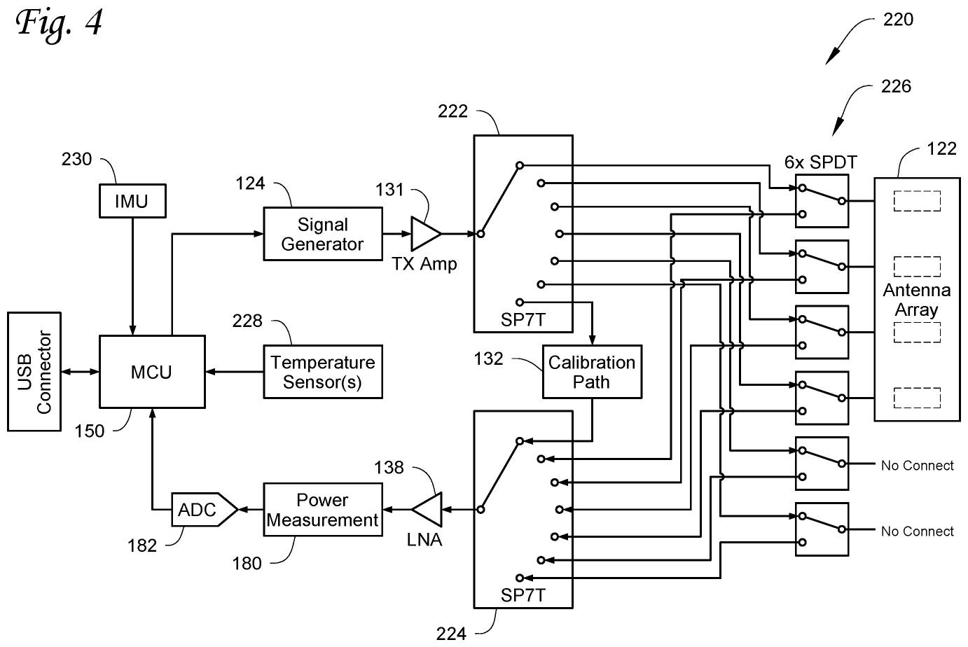

Referring now to FIG. 4, an example of an analyte sensor system 220 is depicted having an antenna array 122 having a plurality of antennas. The array 122 can be similar to the antenna arrays in FIGS. 2 and 3 or it can have a different configuration with a different number of antennas. In this example, the array 122 is depicted as having four antennas (depicted in broken lines), however a smaller or larger number of antennas can be used in the array 122. The system 220 includes transmit side circuitry that includes a radio frequency (RF) signal generator 124 and a transmit amplifier 131. The RF signal generator 124 generates a radio frequency signal to be transmitted. The RF signal generated by the signal generator 124 can have a frequency that is in a range from about 10 kHz to about 100 GHz, or in a range from about 500 MHz to about 3000 MH2. The signal is then amplified by the transmit amplifier 131 to a level appropriate to detect or measure the analyte. The system 220 further includes receive side circuitry that includes an amplifier 138, such as a low noise amplifier, a power detector 180, and an analog to digital converter (ADC) 182. Microcontroller 150 is connected to and controls the signal generator 124, and the detection circuitry of the power detector 180 and the ADC 182. The RF power detector 180 can be any type of detector that is suitable for detecting the RF power output from the amplifier 138. In one non-limiting example, the RF power detector 180 can be a log amplifier. The RF power detector 180 and the ADC 182 can be implemented as discrete circuits/components as illustrated, integrated into the microcontroller 150, or integrated together.

The system 220 further includes a first set of switches including a transmit side switch 222 and a receive side switch 224, and a second set of switches 226 between the switches 222, 224 and the antenna array 122. The switches 222, 224, 226 control which antenna(s) in the array 122 function as transmit antenna(s) and which antenna(s) in the array 122 function as receive antenna(s). The switches 222, 226 direct the signal to be transmitted to any one or more of the antennas of the array 122 to transmit the signal. The switches 224, 226 selectively connect to any one or more of the antennas of the array 122 to act as a receive antenna.

A direct connection between the switches 222, 224 can also be established to form a calibration path 132 that bypasses the antenna array 122 to directly connect the transmit side circuitry to the receive side circuitry. The calibration path 132 represents the measurement of a known quantity for the purposes of system calibration and test.

The system 220 can also include one or more temperature sensors 228 that sense the temperature of the RF circuitry, such as one or more individual components thereof, and/or sense the temperature of the user/patient and/or sense the temperature of the analyte and/or sense ambient temperature where the analyte sensor is located. Changes in temperature may impact sensor performance or how certain analytes interact with the sensor and/or the transmitted signal. The sensed temperature(s) can be used to post-process (i.c., correct) the data collected by the system 220, such as to account for temperature-related signal drift, and/or used to adjust operation of the system 220. The temperature data may also be used to: inform or augment data collection; inform or augment how the received signal data is processed/analyzed; and/or inform or augment other elements of the sensor system 220 such as safety, overheating, and battery health.

The system 220 is also depicted as including one or more motion sensors 230. The motion sensor(s) 230 senses motion of the system 220 which may be motion of a component of the system 220 relative to the user, motion of the entire system 220 relative to the user, or motion of the system 220 (or component of the system 220) together with the user. The motion sensor 230 can be at least one sensor from the group of an accelerometer, a gyroscope, an inertial measurement unit, or other sensor capable of sensing motion. Motion data obtained by the motion sensor 220 can be used for a number of purposes. For example, the motion data can be used to filter the analyte data obtained at the time of the movement to obtain better, more reliable analyte data results. Motion (whether of a sensor component, the entire sensor, or of the user) may cause noise, artifacts, or other errors in the signals received by the receive antenna(s). That noise, artifacts, or errors can be filtered out of the analyte data. In another embodiment, the motion data can be used to determine if the analyte data obtained at the time of the movement should be thrown out, not relied upon, or not presented to the user based on the analyte data being too inaccurate due to the movement. In another embodiment, the motion data can be used to trigger an analyte reading (for example trigger a reading if the user is not moving, or trigger a reading if the amount of motion is at or below a threshold, or trigger a reading if the amount of motion is at or above a threshold), or prevent an analyte reading (for example prevent a reading if the user is moving, prevent a reading if amount of motion is at or below a threshold, or prevent a reading if the amount of motion is at or above a threshold). The motion data can also be used as follows: If the user is moving in a certain way, change the sensor parameters to optimize sensor performance for that use case/application; if a user is moving in a certain way, change the power levels to optimize battery life and device usability over time; if the sensor is moving in an abnormal way, alert the user or other entity of a potential error; if the user is moving in an abnormal way, alter the user or other entity of a potential risk; if the user is moving in an abnormal way, and the sensor detects concerning levels of a certain analyte, call an ambulance, doctor or other caregiver.

The terminology used in this specification is intended to describe particular embodiments and is not intended to be limiting. The terms “a,” “an,” and “the” include the plural forms as well, unless clearly indicated otherwise. The terms “comprises” and/or “comprising,” when used in this specification, specify the presence of the stated features, integers, steps, operations, elements, and/or components, but do not preclude the presence or addition of one or more other features, integers, steps, operations, elements, and/or components.

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

1. A non-invasive analyte sensor system, comprising:
an antenna that is configured to detect an electromagnetic wave in a radio or microwave frequency range that results from transmission of a transmit signal in the radio or microwave frequency range into a target containing at least one analyte;
a receive circuit that is electrically connectable to the antenna to convert the electromagnetic wave into one or more signals representing the at least one analyte; and
a motion sensor.

2. The non-invasive analyte sensor system of claim 1, wherein the motion sensor comprises an accelerometer, a gyroscope, or an inertial measurement unit.

3. The non-invasive analyte sensor system of claim 1, wherein the at least one analyte comprises glucose, alcohol, white blood cells, oxygen, or luteinizing hormone.

4. The non-invasive analyte sensor system of claim 1, further comprising a housing configured to contain the motion sensor.

5. The non-invasive analyte sensor system of claim 4, wherein the motion sensor is configured to sense motion of the housing relative to the target.

6. The non-invasive analyte sensor system of claim 4, wherein the motion sensor is configured to sense motion of the housing together with the target.

7. The non-invasive analyte sensor system of claim 1, further comprising a controller configured to process analyte data contained in the one or more signals representing the at least one analyte in view of motion data obtained by the motion sensor.

8. The non-invasive analyte sensor system of claim 7, wherein the controller is configured to process the analyte data to filter analyte data obtained at a time of movement based on the motion data at the time of movement.

9. The non-invasive analyte sensor system of claim 7, wherein the controller is configured to process the analyte data to determine that an analyte measurement should not be utilized based on the motion data.

10. The non-invasive analyte sensor system of claim 9, wherein the controller is configured to determine that an analyte measurement should not be utilized if it is determined that the analyte measurement is too inaccurate based on the motion data.

11. The non-invasive analyte sensor system of claim 1, further comprising a controller configured to utilize motion data obtained by the motion sensor to trigger an analyte measurement using the antenna.

12. The non-invasive analyte sensor system of claim 1, further comprising a controller configured to utilize motion data obtained by the motion sensor to prevent an analyte measurement using the antenna.

13. A non-invasive analyte sensor system, comprising:
an antenna that is configured to detect an electromagnetic wave in a radio or microwave frequency range that results from transmission of a transmit signal in the radio or microwave frequency range into a target containing at least one analyte;
a receive circuit that is electrically connectable to the antenna to convert the electromagnetic wave into one or more signals representing the at least one analyte; and
a temperature sensor.

14. The non-invasive analyte sensor system of claim 13, wherein the temperature sensor is configured to sense a temperature of one or more of the antenna and the receive circuit.

15. The non-invasive analyte sensor system of claim 13, wherein the temperature sensor is configured to sense a temperature of the target.

16. The non-invasive analyte sensor system of claim 13, wherein the temperature sensor is configured to sense a temperature of the at least one analyte.

17. The non-invasive analyte sensor system of claim 13, wherein the temperature sensor is configured to sense an ambient temperature.

18. The non-invasive analyte sensor system of claim 13, further comprising a controller configured to adjust operation of the antenna based on temperature data obtained by the temperature sensor.

19. The non-invasive analyte sensor system of claim 13, further comprising a controller configured to process the one or more signals representing the at least one analyte based on temperature data obtained by the temperature sensor.

20. The non-invasive analyte sensor system of claim 19, wherein the controller is configured to process the one or more signals representing the at least one analyte based on the temperature data obtained by the temperature sensor to account for temperature-related signal drift.
